(19) United States
(12) Patent Application Publication
(10) Pub. No.: US 2025/0176894 A1
(43) Pub. Date: Jun. 5, 2025

(54) ARRYTHMIA DETECTION REPROGRAMMING RECOMMENDATION ALGORITHM

(71) Applicant: Cardiac Pacemakers, Inc., St. Paul, MN (US)

(72) Inventors: Abhijit Rajan, Shoreview, MN (US); David L. Perschbacher, Blaine, MN (US)

(21) Appl. No.: 18/952,210

(22) Filed: Nov. 19, 2024

Related U.S. Application Data
(60) Provisional application No. 63/604,724, filed on Nov. 30, 2023.

(51) Int. Cl. A61B 5/33; A61B 5/00; A61B 5/352; A61B 5/361; G16H 40/67; G16H 50/20

(52) U.S. Cl. CPC A61B 5/33; A61B 5/0006; A61B 5/352; A61B 5/361; A61B 5/4836; A61B 5/7203; G16H 40/67; G16H 50/20

(57) ABSTRACT

A system and method for optimizing arrhythmia detection in an ambulatory medical device is provided. Physiological signal data obtained by the medical device is analyzed using one or more machine learning models trained to detect different arrhythmia types. When an arrhythmia episode is detected in the analyzed physiological data that was missed by the device's own detection algorithms, the system automatically generates a recommendation to adjust the device's detection parameters, such as by increasing detection sensitivity, to improve future performance. Periodically re-evaluating collected physiological data with advanced machine learning techniques in this manner enables closed-loop optimization of the device's arrhythmia detection over time. This enhances the accuracy of arrhythmia diagnosis compared to relying solely on the device's static detection settings.

### CLAIM OF PRIORITY

This application claims the benefit of U.S. Provisional Application No. 63/604,724, filed on Nov. 30, 2023, which is hereby incorporated by reference in its entirety.

### TECHNICAL FIELD

The present application relates generally to medical devices and more particularly to systems, devices, and methods, which leverage artificial intelligence (AI), including machine learning models, to generate recommendations regarding the reprogramming of an ambulatory medical device, such as one that may be configured to detect arrythmia.

### BACKGROUND

Ambulatory medical devices, including implantable cardiac devices and wearable cardiac devices, allow for continuous monitoring of heart rhythms and other physiological signals to facilitate detection of arrhythmias, heart failure exacerbations, and other cardiac conditions. Some implantable cardiac devices use sensors embedded directly into the heart tissue to gather detailed electrogram (EGM) signal data reflecting cardiac electrical activity. Other implantable devices, such as subcutaneous medical devices are positioned under the skin and use electrodes to sense cardiac data without direct cardiac contact. Wearable cardiac monitoring devices are external monitors that are worn on the body, usually as a patch or chest strap, and use surface electrodes to non-invasively obtain electrocardiogram (ECG) waveforms for arrhythmia detection without implanted or subcutaneous electrodes making direct cardiac contact. In each case, embedded algorithms analyze the incoming physiological signal data in real-time to identify pathological events based on heart rate irregularities, morphology patterns, timing intervals, variability metrics, noise discrimination, and other characteristics. When specific criteria are met indicating an episode of a targeted condition like atrial fibrillation or ventricular tachycardia, data relating to the detected episodes is stored in device memory and later communicated to a device of a physician, to guide treatment.

### SUMMARY

Disclosed herein are systems and methods for generating a recommendation to reprogram an ambulatory medical device. In some examples, the subject matter involves a method for generating a reprogramming recommendation for an ambulatory medical device, including a computing device having one or more processors and memory for storing executable instructions, to perform the method. The method involves receiving over a network physiological signal data obtained by an ambulatory medical device and then processing the received physiological signal data by inputting the physiological signal data into one or more pre-trained machine learning models. Each of the one or more pre-trained machine learning models has been trained to generate an output indicating whether the physiological signal data represents an arrhythmia episode of a particular type. Upon obtaining an output from the one or more pre-trained machine learning models indicating a detected arrythmia episode of a first type, the method involves determining that an on-device arrythmia detection algorithm of the ambulatory medical device did not detect a corresponding arrythmia episode of the first type. As a result of determining that the on-device arrythmia detection algorithm of the ambulatory medical device did not detect a corresponding arrythmia episode of the first type, a reprogramming recommendation for the ambulatory medical device is generated.

In an example, the ambulatory medical device is programmed to operate in a first sensitivity mode of a plurality of sensitivity modes with each sensitivity mode corresponding with a first set of predefined threshold values for use by an on-device arrythmia detection algorithm in detecting arrythmia episodes. The reprogramming recommendation is a recommendation to program the ambulatory medical device to operate using a second sensitivity mode, where the second sensitivity mode has at least one predefined threshold value for use by the on-device arrythmia detection algorithm that is lower than a corresponding predefined threshold value for the first sensitivity mode. As a result, the new sensitivity mode provides increased sensitivity for detecting arrhythmia episodes compared to the first sensitivity mode.

In an example, which may be combined with other examples, the on-device arrythmia detection algorithm of the ambulatory medical device operates in two stages comprising a detection stage and a confirmation stage. Accordingly, during the detection stage, the on-device arrythmia detection algorithm analyzes incoming physiological signal data and identifies candidate arrythmia events based on the first set of predefined threshold values associated with the first sensitivity mode. When a candidate arrythmia event is identified during the detection stage, the ambulatory medical device begins recording and storing a segment of the physiological signal data corresponding to the time of the initial candidate arrythmia event detection. During a confirmation stage, the on-device arrythmia detection algorithm analyzes the stored segment of physiological signal data corresponding to the candidate arrythmia event to confirm whether the event meets criteria for a confirmed arrythmia episode based on the first set of predefined detection algorithm threshold values associated with the first sensitivity mode. The changing of the programming of the ambulatory medical device from the first sensitivity mode to the second sensitivity mode results in changing to a second set of predefined detection algorithm threshold values used by one or both of the detection stage and confirmation stage.

In an example, which may be combined with other examples, the physiological signal data obtained by the ambulatory medical device is stored locally on the ambulatory medical device until a wireless connection is established between the ambulatory medical device and an intermediary device. Upon establishing the wireless connection with the intermediary device, the physiological signal data is transmitted from the ambulatory medical device to the intermediary device and then transmitted from the intermediary device to a computing device over a network.

In an example, which may be combined with other examples, the computing device performing the method processes the received physiological signal data by inputting the physiological signal data into a trained machine learning model to generate by the trained machine learning model a plurality of confidence scores. Each confidence score indicates a likelihood that the physiological signal data represents an arrhythmia episode of a particular type of arrythmia. For a first type of arrythmia, the confidence score is compared to a confidence threshold to determine whether an episode of the first arrhythmia type is detected in the physiological signal data. This may be repeated for other arrythmia types.

In another example, which may be combined with other examples, the computing device performs processing of the received physiological signal data by inputting the physiological signal data into each of a plurality of trained machine learning models, each trained machine learning model trained to detect a different arrhythmia type. The output obtained from each of the plurality of trained machine learning models indicates whether the physiological signal data represents an episode of the arrhythmia type that the model is trained to detect. In some examples, the method involves, determining that the physiological signal data represents an episode of the first arrhythmia type based on the output of the one of the plurality of trained machine learning models trained to detect the first arrhythmia type.

In yet another example, which may be combined with other examples, a type of arrhythmia episode is an atrial fibrillation episode and the reprogramming recommendation is a recommendation to reprogram the ambulatory medical device to use a sensitivity setting having predefined threshold values for an on-device arrythmia detection algorithm that are more sensitive for detecting atrial fibrillation episodes.

In yet another example, which may be combined with other examples, the increased sensitivity for detecting atrial fibrillation episodes by the on-device arrythmia detection algorithm is achieved by modifying one or more of: i) decreasing an R-R interval irregularity threshold used by the ambulatory medical device to declare an atrial fibrillation episode, ii) decreasing a density index threshold calculated from R-R intervals that must be satisfied to declare an atrial fibrillation episode, and iii) decreasing a minimum atrial fibrillation episode duration threshold.

In another example, which may be combined with other examples, the output from the one or more machine learning models indicates that the physiological signal data contains an arrythmia episode comprising one of premature ventricular contractions (PVCs) or premature atrial contractions (PACs). Based on an indication of PVCs/PACs in the physiological signal data, the reprogramming recommendation comprises modifying one or more predefined threshold values used by an on-device arrythmia detection algorithm of the ambulatory medical device to detect an episode of PVC/PAC.

In another example, which may be combined with other examples, the output from the one or more machine learning models indicates that the physiological signal data contains T-wave oversensing (TWOS). Based on the indication of TWOS in the physiological signal data, the reprogramming recommendation comprises a recommendation to program the ambulatory medical device to operate with a sensitivity mode having a different refractory period to avoid oversensing of T-waves.

In another example, which may be combined with other examples, the generating of the reprogramming recommendation comprises determining that the output from the one or more machine learning models indicates detection of the arrhythmia episode of the first type a predetermined plurality of times. Accordingly, the reprogramming recommendation is generated only after the predetermined plurality of times that the arrhythmia episode of the first type is detected by the one or more machine learning models.

In another example, which may be combined with other examples, the generating of the reprogramming recommendation comprises incrementing a counter each time the output from the one or more machine learning models indicates detection of the arrhythmia episode of a first type. The subject matter involves determining that the counter exceeds a threshold number of days, wherein the counter indicates detection of the arrhythmia episode of the first type on consecutive days. The reprogramming recommendation is generated only after the counter indicates that the arrhythmia episode of the first type is detected on the consecutive days.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a block diagram illustrating an example of an ambulatory medical device, consistent with some embodiments.

FIG. 2 is a block diagram illustrating an example patient management system, consistent with some embodiments.

FIG. 3 is a diagram illustrating an example of a machine learning model that is trained to receive, as input, physiological signal data obtained by an ambulatory medical device, and to generate an output indicating a likelihood of the physiological signal data representing an arrythmia episode of a particular type, according to some embodiments.

FIG. 4 is a flow diagram illustrating method operations for a method, performed by a medical ambulatory device, to obtain and transmit physiological signal data for analysis by a remote computer system, according to some embodiments.

FIG. 5 is a flow diagram illustrating method operations for a method, performed by a computer system, to generate a reprogramming recommendation for an ambulatory medical device, according to some embodiments.

FIG. 6 is a diagram illustrating an example computing device configured to apply one or more trained machine learning models to physiological signal data obtained by an ambulatory medical device, and to generate a reprogramming recommendation for the ambulatory medical device, consistent with some embodiments.

### DETAILED DESCRIPTION

Ambulatory medical devices, like implantable cardiac devices, subcutaneous cardiac monitors, and wearable cardiac monitors use algorithms to continuously analyze physiological signal data to identify different types of arrhythmias. Implantable cardiac monitors obtain detailed cardiac electrical data through sensing electrodes embedded directly into the heart tissue that gather electrogram (EGM) waveforms reflecting electrical activity over time. Subcutaneous cardiac devices are positioned under the skin and use electrodes to sense cardiac signal data without direct cardiac contact, capturing electrocardiogram (ECG) waveforms. Wearable cardiac monitoring devices are external monitors that are worn on the body, usually as a patch or chest strap, and use surface electrodes to non-invasively obtain electrocardiogram (ECG) waveforms for arrhythmia detection without implanted or subcutaneous electrodes making direct cardiac contact. In each case, pattern recognition algorithms evaluate characteristics of the incoming physiological signal data including timing, morphology, rate, regularity, and other features. The ambulatory medical devices identify potential arrhythmia episodes when the received physiological signal data matches or otherwise satisfies predefined pattern matching criteria for conditions like atrial fibrillation, ventricular tachycardia, bradycardia, asystole, and more.

Many ambulatory medical devices, including cardiac monitoring devices, are capable of detecting different arrhythmia types or pathologies. In many instances, the arrythmia detection algorithms of a cardiac monitoring device are configurable to operate at different sensitivity settings or sensitivity modes, for detecting various arrhythmia types. For instance, a clinician may program an ambulatory cardiac device by specifying one of a number of predetermined sensitivity settings or modes (e.g., “least”, “less”, “balanced”, “more”, and “most), where the selected sensitivity setting has an impact on various thresholds used by an on-device arrythmia detection algorithm. Generally, a higher sensitivity setting lowers the value of one or more of the thresholds required to detect an arrhythmia, while a lower sensitivity setting raises the arrhythmia detection thresholds.

Many ambulatory medical devices, including cardiac monitoring devices, have inherent constraints that limit their ability to perform some state-of-the-art arrythmia detection algorithms directly on the device. In the case of implantable cardiac monitoring devices, the cardiac monitoring device resides inside the patient's body for extended periods without maintenance. The embedded battery of an implantable device has a finite lifespan and cannot be easily replaced or recharged once implanted. Moreover, the small size of an implantable device restricts processing power and data storage capabilities. To conserve power for years of use, the processors have limited speed and memory. The on-device firmware and arrythmia detection algorithms cannot be easily updated after the device is implanted. Finally, stringent reliability requirements apply to all functions performed within the implantable device since it cannot be easily accessed for repairs. Due to these size, power, updatability, and reliability constraints, running computationally intensive arrythmia detection algorithms directly on the implantable device is not feasible.

Due to constraints like limited battery life and processing power, some ambulatory medical devices like implantable cardiac monitors use a two-stage approach for arrhythmia detection. In a first stage, the device continuously monitors the cardiac rhythm in real-time without recording or storing data by using infrequent sampling or low resolution to minimize power usage. In some instances, the programmed sensitivity setting or mode may impact detection thresholds at this stage. For example, a higher sensitivity setting or mode for the device may lower a predefined threshold for detecting an R-wave, allowing more subtle waveform features to be sensed. When the arrythmia detection algorithm identifies a potential arrhythmia triggering event based on criteria like a certain number of irregular beats, the device switches to the second stage—the confirmation stage—where physiological signal data is recorded, and additional algorithmic confirmation checks are performed.

In the second stage, the device begins actively recording and storing the physiological signal data from the time of the initial triggering event detection. The amount of data captured in the confirmation stage may depend on a configuration setting of the device. For example, the device may be configured to detect specific types of arrhythmias, and depending upon which type is targeted for detection, the amount of recorded data that is stored may vary. After storing the relevant signal data, the device applies additional algorithmic checks tailored to the suspected arrhythmia type, such as evaluating beat morphology, noise levels, rate regularity, and patterns. Here again, the device's programmed sensitivity setting or mode may impact one or more thresholds used in one or more of the algorithmic confirmation checks that are part of the confirmation stage for a specific arrythmia type.

Despite optimizations like the two-stage detection approach, the inherent constraints of ambulatory medical devices prevent implementing state-of-the-art pattern recognition algorithms directly on the device. For example, complex machine learning models require substantial processing power and continual re-training as new data is collected. This is not feasible given the fixed processing capabilities and inability to easily update firmware on implanted devices. As a result, the device must be pre-configured by selecting from a limited set of settings, like arrhythmia types and sensitivity modes. However, static configuration can lead to missed arrhythmia episodes or false detections over time as cardiac conditions evolve. The preset parameters may become suboptimal for identifying certain arrhythmia types in a given patient. Missed episodes and false positives can misinform therapy decisions or delay further diagnosis.

Having recognized the technical problems associated with misdetections resulting from suboptimal preset configurations in ambulatory medical devices, the inventors have devised an improved approach and system. Consistent with some embodiments, the improved approach involves periodically collecting physiological signal data from an ambulatory medical device, for example, according to a predetermined schedule. For example, the ambulatory medical device is configured to automatically obtain and store a segment of physiological signal data according to some preset schedule. The schedule may be one or more times per day, at set times. Alternatively, the recording of the physiological data for external analysis could be performed at randomly selected times. This stored physiological data is then transferred from the device to an external computing system for analysis by one or more machine learning models that are more computationally intensive and have higher detection performance than the on-device arrythmia detection algorithms. The machine learning models can identify subtle patterns in the physiological signal data that may indicate arrhythmia episodes that were missed by the device's own pattern recognition algorithms. Based on the analysis, the external system's machine learning models can identify arrhythmia episodes in the collected physiological data that may not have been detectable by either the device's arrythmia detection algorithms, given the current sensitivity setting, or by a clinician visually analyzing the data.

When suboptimal performance of the ambulatory medical device is identified, the external computer system can generate personalized recommendations to adjust the device sensitivity setting or mode, which impacts various predefined thresholds used by the on-device arrythmia detection algorithms in identifying arrythmia episodes. Here, sub-optimal performance of the device's arrythmia detection algorithm is determined based on a machine learning model detecting one or more arrhythmia episodes in one or more instances of the collected physiological signal data, where the on-device arrythmia detection algorithm failed to detect a corresponding arrythmia episode. For example, the device may be configured to detect atrial fibrillation operating at a sensitivity mode programmed to “low”. In this setting, the device may miss episodes of paroxysmal AF in the patient's changing heart rhythms. An external computing device that applies one or more machine learning models to physiological signal data periodically obtained by the device may detect these missed AF episodes, indicating the device sensitivity needs to be increased to “medium” or “high” to improve future AF detection. This closed-loop, data-driven approach allows enhancing detection accuracy in an ongoing manner for individual patients as their cardiac health evolves.

Consistent with some embodiments, a reprogramming recommendation may involve a recommendation to modify or reprogram the device to use a sensitivity setting or mode different from the current sensitivity setting or mode. With some embodiments, each sensitivity setting, or sensitivity mode is associated with one or more predefined threshold values used by an arrythmia detection algorithm to detect an arrythmia event of a particular type. Accordingly, when a device is reprogrammed to operate in a new sensitivity mode, one or more of the predefined threshold values used by an arrythmia detection algorithm will change, thereby increasing or decreasing the sensitivity for detecting a particular arrythmia type. The reprogramming recommendation may be presented via a user interface of a software application to a clinician, who will undertake the task of reprogramming the device for a patient. The clinician can then evaluate the recommendation and reprogram the ambulatory medical device accordingly. This allows the clinician to validate any proposed changes to the device based on their expert judgment.

In alternative embodiments, the recommendation could be communicated directly to the patient, rather than requiring clinician involvement. For example, the recommendation may be sent to a mobile application or dedicated intermediate patient interface device that integrates with the ambulatory medical device. Technically, this would allow the patient to directly reprogram the device based on the system's reprogramming recommendation. However, any direct patient reprogramming of an implanted medical device would need to comply with safety regulations and clinical guidelines governing these practices. Additional safeguards and clinician oversight would likely be required depending on the implementation.

Embodiments of the present invention provide numerous technical advantages for optimizing arrhythmia detection in ambulatory medical devices. By periodically evaluating collected physiological signal data using advanced machine learning models resident on an external system, embodiments of the invention enable closed-loop optimization of the device's detection algorithms over time. This allows enhancing arrhythmia diagnosis accuracy compared to relying solely on the device's static detection settings programmed at implantation. The external machine learning analysis can identify subtle arrhythmia patterns missed by the device's algorithms and recommend adjustments to improve sensitivity when appropriate. The system adapts the device's detection parameters to the individual patient as their cardiac health evolves. This results in more timely and accurate arrhythmia diagnosis without requiring constant manual reprogramming by clinicians. The invention reduces workload for clinicians while providing a competitive advantage over other ambulatory cardiac monitoring devices that lack this automated, data-driven optimization capability. Other aspects and advantages of the present invention are presented below in connection with the description of the several drawings.

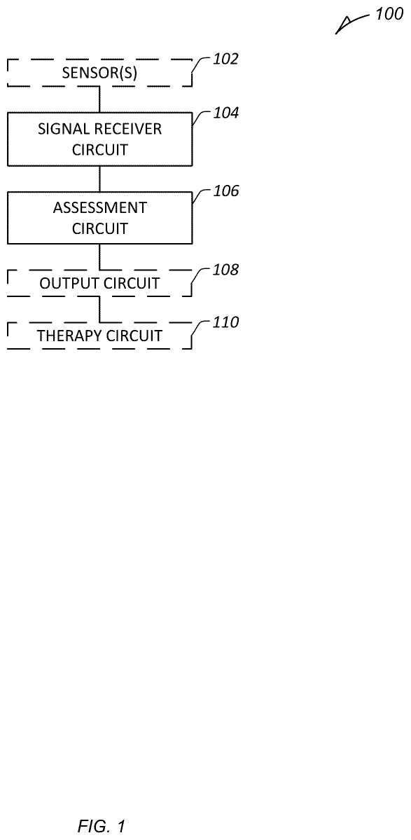

FIG. 1 illustrates an example system 100 (e.g., a medical device system), consistent with some embodiments. In an example, one or more aspects of the system 100 can be a component of, or communicatively coupled to, a medical device, such as an ambulatory medical device (AMD), an implantable medical device (IMD), an insertable medical device such as an insertable cardiac monitor (ICM), or a wearable medical device. The system 100 can monitor, detect, or in some instances treat various physiological conditions of the body. For example, the system can address cardiac conditions associated with the heart's reduced ability to sufficiently pump blood to the body. Such cardiac conditions can include heart failure, arrhythmias, dyssynchrony, and more. In certain examples, the system 100 can also be configured to provide electrical stimulation or other therapies or treatments to the patient to address these conditions.

The system 100 can include a single medical device or multiple medical devices implanted in a patient's body or otherwise positioned on or about the patient. These devices monitor patient physiological information using one or more sensors, such as sensor 102. For example, the sensor 102 can include: a respiration sensor configured to receive respiration information (e.g. respiratory rate, tidal volume); an acceleration sensor (e.g. an accelerometer, microphone) configured to receive cardiac acceleration information (e.g. cardiac vibration, pressure waveform, heart sound, endocardial acceleration, activity, posture); an impedance sensor configured to receive impedance information; a cardiac sensor configured to receive cardiac electrical information; an activity sensor configured to receive physical motion information (e.g. activity, steps); a posture sensor configured to receive posture or position information; a pressure sensor configured to receive pressure information; a photoplethysmography sensor; a chemical sensor (e.g. electrolyte, pH, anion gap sensor); a temperature sensor; a skin elasticity sensor; or other sensors configured to receive patient physiological information.

The system 100 includes a signal receiver circuit 104 and an assessment circuit 106. The signal receiver circuit 104 receives physiological information (e.g., physiological signal data) from one or more sensors 102 positioned on or in the patient. The assessment circuit 106 analyzes the data obtained from the signal receiver circuit 104. The assessment circuit 106 determines physiological parameters, stratifiers, or changes in patient condition based on the received physiological information. For example, the assessment circuit can detect indications of dehydration, respiratory issues, cardiac conditions like heart failure or arrhythmia, sleep disordered breathing, and more. The physiological information analyzed can include cardiac electrical information, impedance, respiration, heart sounds, activity, posture, temperature, or other data.

In some examples, the assessment circuit 106 can aggregate data from multiple sensors or devices. It may detect various events by analyzing information from each sensor separately or in combination. The assessment circuit 106 can update detection status for one or more patients based on the sensor information. It can also transmit messages or alerts to remote devices indicating a detection has been made or information has been stored/transmitted. This allows additional systems or processes to use the detection data or information for further review.

In some examples, detecting changes in a patient's condition requires first establishing a baseline level using physiological information from one or more sensors. Subsequent detection of deviation from this baseline can indicate improving or worsening condition. However, in other examples, the amount of variation or change in physiological information over different time periods can also detect risks of adverse medical events. This can predict or stratify the patient's risk of a future adverse event like heart failure. It does so by detecting changes, with or without a baseline.

Changes in different physiological information can be aggregated and weighted based on patient-specific factors. In certain examples, they are compared to thresholds with clinical sensitivity/specificity for a population with a certain condition (e.g., heart failure). The time periods assessed may include daily values, short-term averages (daily values over several days), or long-term averages (daily values over short-term periods or more days, sometimes non-overlapping days).

In some examples, the system may be programmable, and reprogrammable, to operate in one of a number of predefined sensitivity settings or modes. For example, the system 100 is programmable by a clinician to operate using one of several predefined sensitivity settings or modes for detecting various arrhythmia types. Each sensitivity mode corresponds to a specific set of detection algorithm threshold values tailored to that mode. For example, a “high” sensitivity mode will have lower thresholds for criteria like beat irregularity, noise levels, and R-R interval variation compared to a “low” sensitivity mode.

To program the sensitivity mode, the clinician establishes a wireless connection between the system 100 and an external programming device. This may involve inductive near-field communication, radio frequency, or other wireless standards suitable for medical devices. The external programming device provides a user interface allowing the clinician to select the desired sensitivity mode and wirelessly transmit the programming commands to the system 100. The programming commands are received by a wireless module (not shown) within the system 100 and used to update the configuration settings that control the detection algorithm thresholds, and specifically the threshold values for use by the arrythmia detection algorithms.

After reprogramming, the system 100 operates using the updated detection algorithm thresholds associated with the newly selected sensitivity mode. This allows the clinician to adjust the sensitivity as needed for an individual patient over time as their cardiac health evolves to optimize arrhythmia detection accuracy. The ability to reprogram sensitivity modes provides flexibility compared to systems with static, unchangeable settings.

The system 100 includes an output circuit 108 to provide outputs to the user or the user's device. For example, it can output a score, trend, alert, recommendation or other indication via a display or user interface. In other cases, the output circuit 108 can provide outputs to control another circuit, machine, or process. For instance, it can connect to a therapy circuit 110 like a cardiac resynchronization therapy (CRT) circuit, chemical therapy circuit, or stimulation circuit. This allows it to adjust or halt therapy from a medical device or drug delivery system. It can also alter processes of other medical device system aspects, such as CRT parameters and drug delivery dosage. The therapy circuit 110 may include stimulation control, cardiac stimulation, neural stimulation, or dosage control circuits. In some examples, the assessment circuit 106 can control the therapy circuit 110. The assessment circuit 106 may also determine the output for the output circuit 108, while the output circuit 108 provides the signals to generate the user interface output.

In some examples, the system 100 is configurable to periodically store segments of physiological signal data in its internal memory, unrelated to identifying a specific triggering event. With the segment of physiological signal data stored, the output circuit 108 establishes a wireless connection with an intermediary device such as a mobile computing device executing a dedicated mobile application, or a dedicated intermediary device purpose built to integrate with the system 100. This periodic connection allows the stored physiological signal data to be transmitted from the system 100 to the intermediary device. The intermediary device then forwards the physiological signal data over a computer network, such as the Internet, to a remote server residing in a secured cloud-based environment. On the remote server, the physiological signal data segments are analyzed by one or more machine learning models to detect arrhythmia episodes that may have been missed by the on-device detection algorithms implemented in the assessment circuit 106. This deeper analysis by more complex algorithms running on the remote server allows for optimization and improvement of the detection thresholds and sensitivity modes programmed into the system 100 over time. The remote server provides a user interface for clinicians to review the physiological signal data and remotely monitor patient status. In cases where one or more arrythmia episodes are detected by the remote server computer, but went undetected by the on-device arrythmia detection algorithm(s), a reprogramming recommendation may be generated and presented to a clinician. Specifically, the reprogramming recommendation may be a recommendation to program the system to operate in a sensitivity mode different from the current sensitivity mode.

A challenge for developing medical devices is managing tradeoffs between battery life versus data collection and processing. Ambulatory medical devices powered by rechargeable or non-rechargeable batteries often operate in a low-power monitoring mode to conserve energy. However, this requires sacrificing data sampling resolution, frequency, processing, storage, and transmission of sensed physiological signal data. For implantable devices with non-rechargeable batteries, it also impacts the replacement surgery frequency.

Ambulatory medical devices can switch to a high-power mode when physiological signal data indicates a potential adverse event. However, by then, valuable data has already been lost and cannot be recovered in the high-power mode. The high-power mode has higher resource requirements like more processing, memory, communication bandwidth, and data transfers.

Inaccurate triggers to switch to high-power mode also unnecessarily drain the battery, reducing usable device lifetime. Accurately detecting physiological events and avoiding unnecessary power mode switching is important to optimize medical device resource usage.

Power mode changes can enable higher resolution sampling or more frequent sampling. For example, the same accelerometer may sense heart sounds and activity in non-overlapping time periods using different sampling rates and power levels. Transitioning to high-power mode could allow continuous heart sound detection throughout, improving detection of cardiac events.

High-power mode also allows recording waveforms and transferring them to a clinician for review. But even stored events consume resources for processing and storage. Careful mode switching is needed to capture relevant physiological data while managing device resources.

In some examples, the system 100 may apply pattern recognition or arrythmia detection algorithms to detect arrhythmia episodes in a two-stage process. The first stage operates as a detection algorithm, continuously monitoring the cardiac rhythm in real-time without recording or storing data. When the detection algorithm identifies a potential arrhythmia event based on predefined triggering criteria, the system switches to the second, confirmation stage.

In the confirmation stage, the system begins recording and storing the cardiac signal data from the time of the initial triggering event detection. The amount and duration of data captured depends on system settings for that arrhythmia type, or the nature of the triggering event. The confirmation algorithm then analyzes the stored signal data to verify the event meets criteria for a confirmed episode. For example, it may evaluate rate, regularity, noise levels, and waveform morphology in the recorded data to differentiate true arrhythmia from false positives. The confirmation checks are tailored to the specific arrhythmia type associated with the initial trigger, such as atrial fibrillation, bradycardia, tachycardia, asystole and more. This two-stage approach allows robust detection while minimizing memory usage.

Consistent with some embodiments, the system 100 is configurable or programmable to detect different arrhythmia types or pathologies. Physicians may program an ambulatory medical device by specifying one of a number of predetermined sensitivity settings or modes (e.g., “least”, “less”, “balanced”, “more”, and “most”). The selected sensitivity setting impacts various algorithm thresholds used by the pattern recognition algorithms, in both the detection and confirmation stages.

For example, for atrial fibrillation detection, the R-R interval variability or density index thresholds used to declare AF could be reduced with increased sensitivity. This allows more subtle irregularity to be detected. The sensitivity setting may also impact sampling resolution-higher sensitivity equates to increased sampling frequency and resolution when applicable. Accordingly, increased sensitivity provides more data for analysis by reducing thresholds and allows detection of more subtle arrhythmia episodes, at the risk of reduced specificity.

The system 100 may include a configuration setting that causes it to obtain a sample of physiological signal data for a predefined duration on a periodic basis. For example, the system could be set to record 30 seconds of cardiac electrical data once per day at a scheduled time, or multiple times throughout a day. This sampled data is temporarily stored in the system's memory until a wireless connection is established with a remote device. At that time, the stored data is transmitted to a remote server for additional analysis. On the remote server, the transferred physiological signal data can be input into one or more machine learning models that have been pre-trained to identify different types of arrhythmias. Having the system periodically transfer snapshots of raw physiological data allows more advanced algorithms on the remote server to evaluate the patient's rhythms over time. This may detect subtle changes missed by the system's real-time detection algorithms, which may be the result of the system being set to operate using a sensitivity setting or mode that is too low. As described in greater detail below, the remote analysis results can then be used to generate a recommendation to reprogram the system to operate at a different sensitivity setting or mode.

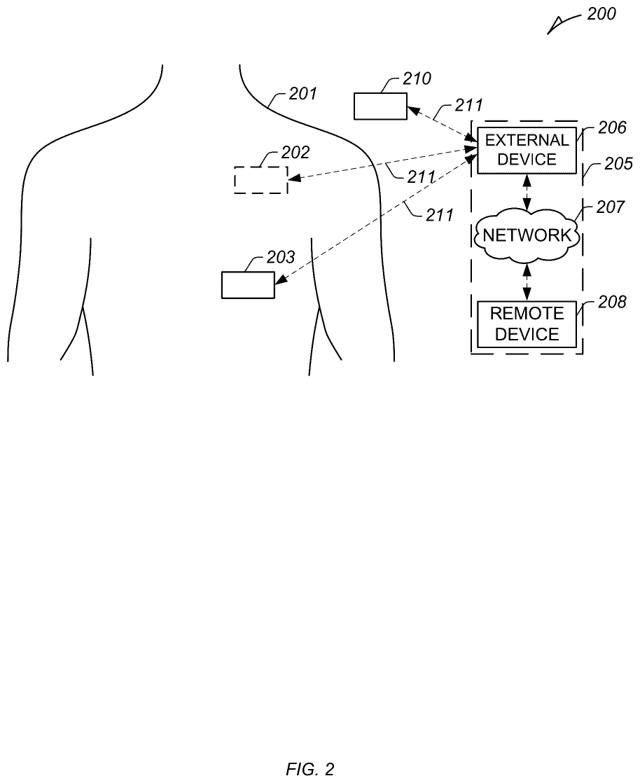

FIG. 2 is a block diagram illustrating an example patient management system 200, consistent with some embodiments. The patient management system 200 can perform a range of activities, including remote patient monitoring and diagnosis of a disease condition. Such activities can be performed proximal to a patient 201, such as in a patient home or office, through a centralized server, such as in a hospital, clinic, or physician office, or through a remote workstation, such as via a secure wireless mobile computing device.

The patient management system 200 can include one or more medical devices, an external system 205, and a communication link 211 providing for communication between the one or more ambulatory medical devices and the external system 205. The one or more medical devices can include an ambulatory medical device (AMD), such as an implantable medical device (IMD) 202, a wearable medical device 203, or one or more other implantable, leadless, subcutaneous, external, wearable, or medical devices configured to monitor, sense, or detect information from, determine physiologic information about, or provide one or more therapies to treat various conditions of the patient 201, such as one or more cardiac or non-cardiac conditions (e.g., dehydration, sleep disordered breathing).

In an example, the implantable medical device 202 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 201. In another example, the implantable medical device 202 can include a cardiac monitoring device implanted, for example, subcutaneously in the chest of the patient 201, the implantable medical device 202 including a housing containing circuitry and, in certain examples, one or more sensors, such as ECG electrodes, an accelerometer, impedance sensor, a temperature sensor, and so forth.

Cardiac rhythm management devices, such as insertable cardiac monitors (ICMs), pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured to detect physiologic information from the patient, or provide one or more therapies or stimulation to the patient.

Implantable cardiac devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 202 can include an assessment circuit configured to detect or determine specific physiologic information of the patient 201, or to determine one or more conditions or provide information or an alert to a user, such as the patient 201 (e.g., a patient), a clinician, or one or more other caregivers or processes, such as described herein. The implantable medical device 202 can alternatively or additionally be configured as a therapeutic device configured to treat one or more medical conditions of the patient 201. The therapy can be delivered to the patient 201 via the lead system and associated electrodes or using one or more other delivery mechanisms. The therapy can include delivery of one or more drugs to the patient 201, such as using the implantable medical device 202 or one or more of the other ambulatory medical devices. In some examples, therapy can include CRT for rectifying dyssynchrony and improving cardiac function in heart failure patients. In other examples, the implantable medical device 202 can include a drug delivery system, such as a drug infusion pump to deliver drugs to the patient for managing arrhythmias or complications from arrhythmias, hypertension, hypotension, or one or more other physiologic conditions. In other examples, the implantable medical device 202 can include one or more electrodes configured to stimulate the nervous system of the patient or to provide stimulation to the muscles of the patient airway, etc.

The wearable medical device 203 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 205 can include a dedicated hardware/software system, such as a programmer, a remote server-based patient management system, or alternatively a system defined predominantly by software executing on a standard personal computer, or mobile computing device. The external system 205 can manage the patient 201 through the implantable medical device 202 or one or more other ambulatory medical devices connected to the external system 205 via a communication link 211. In other examples, the implantable medical device 202 can be connected to the wearable medical device 203, or the wearable medical device 203 can be connected to the external system 205, via the communication link 211. This can include, for example, programming the implantable medical device 202 to perform one or more of acquiring physiologic data, performing at least one self-diagnostic test (such as for a device operational status), analyzing the physiologic data, or optionally delivering or adjusting a therapy for the patient 201. Additionally, the external system 205 can send information to, or receive information from, the implantable medical device 202 or the wearable medical device 203 via the communication link 211. Examples of the information can include real-time or stored physiologic signal data from the patient 201, diagnostic data, such as detection of patient hydration status, hospitalizations, responses to therapies delivered to the patient 201, or device operational status of the implantable medical device 202 or the wearable medical device 203 (e.g., battery status, lead impedance, etc.). The communication link 211 can be an inductive telemetry link, a capacitive telemetry link, or a radio-frequency (RF) telemetry link, or wireless telemetry based on, for example, “strong” Bluetooth or IEEE 602.11 wireless fidelity “Wi-Fi” interfacing standards. Other configurations and combinations of patient data source interfacing are possible.

The external system 205 can include an external device 206 in proximity of the one or more ambulatory medical devices, and a remote device 208 in a location relatively distant from the one or more ambulatory medical devices, in communication with the external device 206 via a communication network 207. Examples of the external device 206 can include a medical device programmer. The remote device 208 can be configured to evaluate collected patient or patient information and provide alert notifications, among other possible functions.

In some examples, the external device 206 serves as an intermediary device for periodically forming a wireless connection with the ambulatory medical device 202 or 203, and then receiving physiological signal data from the ambulatory medical device. For example, the ambulatory medical device may regularly and periodically capture a segment of physiological signal data, for example, according to some predetermined fixed schedule. Upon establishing a wireless connection between the ambulatory medical device and the external device, the stored physiological signal data is transferred to the external device 206, which then relays the data over the network 207 to a remote device 208, such as a cloud-based server computer. At the cloud-based server computer 208, the physiological signal data is provided as input to one or more machine learning models, which have been trained to analyze the physiological signal data to detect various events or episodes, including arrythmia episodes, within the input data.

In an example, the remote device 208 can include a centralized server computer acting as a central hub for collected data storage and analysis from a number of different sources. Combinations of information from the multiple sources, including multiple patients, can be used to make determinations and update individual patient status or to adjust one or more alerts or determinations for one or more other patients. The server can be configured as a uni-, multi-, or distributed computing and processing system. The data received at the remote device 208 can be obtained by the one or more ambulatory medical devices, among other data acquisition sensors or devices associated with the patient 201. The cloud-based server computer can include a memory device to store the data in a patient database. The server can include an alert analyzer circuit to evaluate the collected data to determine if specific alert condition is satisfied. Satisfaction of the alert condition may trigger a generation of alert notifications, or a reprogramming recommendation, such to be provided by one or more human-perceptible user interfaces. In some examples, the alert conditions or recommendations may alternatively or additionally be evaluated by the one or more ambulatory medical devices, such as the implantable medical device. By way of example, alert and reprogramming notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 208 may additionally include one or more locally configured clients or remote clients securely connected over the communication network 207 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 208, including the server and the interconnected clients, may also execute a follow-up scheme by sending follow-up requests to the one or more ambulatory medical devices, or by sending a message or other communication to the patient 201 (e.g., the patient), clinician or authorized third party as a compliance notification.

The communication network 207 can provide wired or wireless interconnectivity. In an example, the communication network 207 can be based on the Transmission Control Protocol/Internet Protocol (TCP/IP) network communication specification, although other types or combinations of networking implementations are possible. Similarly, other network topologies and arrangements are possible.

One or more of the external device 206 or the remote device 208 can output the detected medical events to a system user, such as the patient or a clinician, or to a process including, for example, an instance of a computer program executable in a microprocessor. In an example, the process can include an automated generation of recommendations for reprogramming the sensitivity mode of the medical device, anti-arrhythmic therapy, or a recommendation for further diagnostic test or treatment. In an example, the external device 206 or the remote device 208 can include a respective display unit for displaying the physiologic or functional signals, or alerts, alarms, emergency calls, or other forms of warnings to signal the detection of arrhythmias. In some examples, the external system 205 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

With some examples, when physiological signal data is analyzed using one or more trained machine learning models, and one or more arrythmia episodes are detected, a recommendation to reprogram the medical device may be generated and presented to a clinician via a user interface of the remote device 208, or via a user interface of a software application executing on a client device communicatively connected with the remote device 208. The recommendation to reprogram the medical device may be determined by identifying arrythmia events via the one or more machine learning models that otherwise went undetected by the on-device arrythmia detection algorithms. For example, when the physiological signal data that is periodically obtained and stored by the medical device is analyzed using a machine learning model, and the model output indicates an arrythmia event, the server may attempt to identify whether or not the on-device arrythmia detection algorithm also detected the arrythmia event. When one or more arrythmia events go undetected by the on-device algorithm, but are identified by the external analyzes of the one or more machine learning models, a recommendation to reprogram the device may be generated and communicated to a clinician.

Portions of the one or more ambulatory medical devices or the external system 205 can be implemented using hardware, software, firmware, or combinations thereof. Portions of the one or more ambulatory medical devices or the external system 205 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 210 can be configured to send information to or receive information from one or more of the ambulatory medical devices or the external system 205 using the communication link 211. In an example, the one or more ambulatory medical devices, the external device 206, or the remote device 208 can be configured to control one or more parameters of the therapy device 210. The external system 205 can allow for programming the one or more ambulatory medical devices and can receives information about one or more signals acquired by the one or more ambulatory medical devices, such as can be received via a communication link 211. The external system 205 can include a local external implantable medical device programmer. The external system 205 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), such as in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected atrial fibrillation event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, such as to increase arterial pressure, maintain cardiac output, and to disrupt or reduce the impact of the detected atrial fibrillation event.

In certain examples, physiologic information of a patient can be sensed, such as by one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval. An S4 signal portion can be determined, such as by a processing circuit of the heart sound sensor or one or more other medical devices or medical device components, etc. In certain examples, the S4 signal portion can include a filtered signal from an S4 window of a cardiac interval. In an example, the S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite S1 parameter representative of a plurality of S1 parameters, for example, over a certain time period (e.g., a number of cardiac cycles, a representative time period, etc.).

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient.

In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In an example, the received cardiac acceleration information can include the S4 signal portion occurring between the first and second cardiac features of the patient. In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

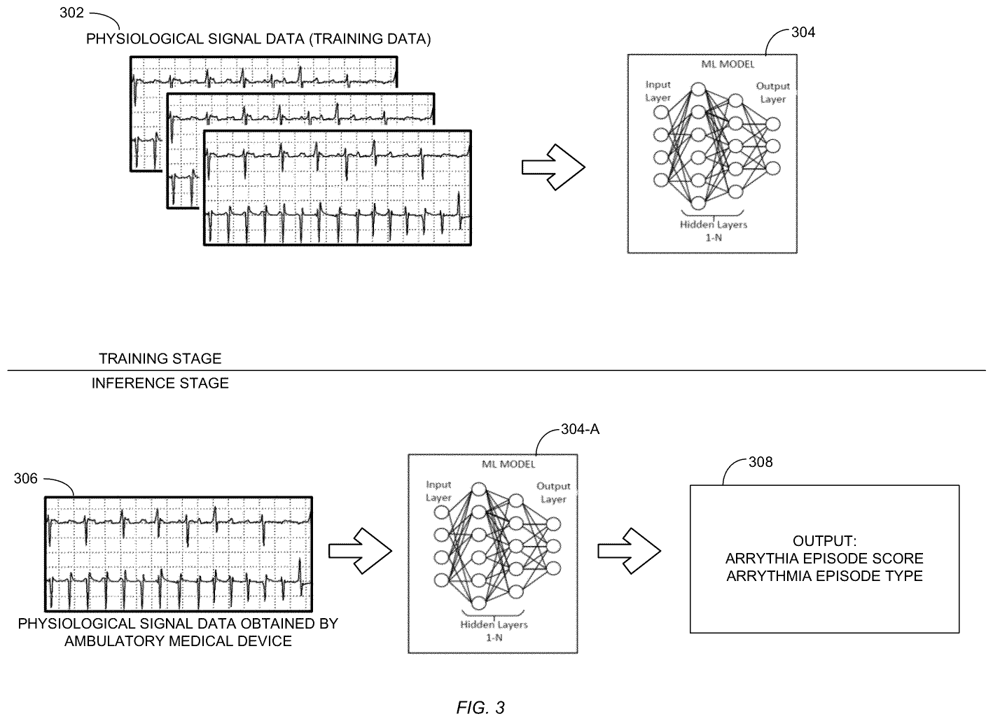

FIG. 3 is a diagram illustrating an example of a machine learning model 304 that is trained to receive, as input, physiological signal data 302 obtained by an ambulatory medical device, and to generate an output indicating a likelihood of the physiological signal data representing an arrythmia episode, according to some embodiments. In the top portion of FIG. 3, a training (or learning) stage is shown, during which a machine learning algorithm is applied with training data 302 to train the machine learning model 304. Here, the training data comprises ECGs or ECG waveforms 302 collected from a population. The training data 302 may also include, for each of the ECGs, respective annotated data (e.g., labels) comprising assessment of physiological parameters, other physiological events, or device operating status for a corresponding ECG. The assessment can be provided by a user, such as based on direct measurements of physiological parameters by application-specific sensors, or diagnostic tests using application-specific instruments (e.g., echocardiograms or other diagnostic imaging tests, electrical mapping for diagnosing cardiac arrhythmias, invasive glucose test via finger prick, glucose strip, or continuous glucose monitors). Accordingly, in some examples, other physiological data, like heart rate, heart sounds, and blood pressure may also be provided as training data, in combination with the ECG waveform data, to train a machine learning model. However, the ECG waveform contains the most direct and comprehensive data for automated arrhythmia detection using machine learning models. The sequential nature and morphological patterns make ECG ideal for machine learning model input.

The machine learning model 304 can be trained using supervised learning, unsupervised learning, or reinforcement learning. Examples of machine learning model architectures and algorithms may include, for example, decision trees, neural networks, support vector machines, or deep neural networks, etc. Examples of deep neural networks can include a convolutional neural network (CNN), a recurrent neural network (RNN), a deep belief network (DBN), a long-term and short-term memory (LSTM) network, a transfer learning network, or a hybrid neural network comprising two or more neural network models of different types or different model configurations. The training of the machine learning model may be performed continuously or periodically, or in near real time as additional patient data are made available. The training involves algorithmically adjusting one or more model parameters, until the model being trained satisfies a specified training convergence criterion. The trained machine learning model 304 can establish a correspondence between ECGs and an arrythmia episode.

In some examples, a machine learning model can be trained to analyze physiological signal data and detect different types of arrhythmias. To train such a model, a dataset is assembled containing sample ECG waveform segments from a population. These ECG samples are annotated by medical experts to label each segment with the type of arrhythmia it represents, if any. This annotated training dataset is then used to train the machine learning model using a supervised learning approach.

The model is trained by algorithmically adjusting its internal parameters to map the input ECG signals to the expert-provided arrhythmia labels. Various machine learning algorithms can be used, including neural networks, support vector machines, decision trees, etc. Deep learning neural network architectures that perform feature extraction are well-suited for ECG analysis. The training process continues until the model achieves a high accuracy in classifying arrhythmias based on the training data.

In some examples, one machine learning model may be trained to generate a plurality of outputs based on a single instance of input data, where each output is a confidence score associated with and indicating a likelihood of the input data representing an arrythmia episode of a particular type. This type of machine learning model is generally referred to as a multi-class classifier, where each confidence score indicates the likelihood that the input data is a member of a particular class. Alternatively, consistent with some examples, multiple machine learning models may be trained, where each individual machine learning model is trained to generate a single output, based on the input data, where the output indicates a probability that the input data represents an arrythmia episode of a particular type. In this case, the output of each model is compared to a relevant threshold for that model, and when a specific output for a specific model exceeds its respective threshold, the output is determined to indicate the occurrence of an arrythmia episode of a particular type, as associated with the specific model.

The training and utilization of machine learning models for arrhythmia detection will depend on the specific type of ambulatory medical device being used and the nature of the physiological signal data it collects. For example, some ambulatory cardiac devices like implantable cardiac monitors obtain detailed electrogram (EGM) signals by using electrodes embedded directly in the heart tissue. EGM signals reflect the cardiac electrical activity over time in a very detailed manner. To train a machine learning model for arrhythmia detection using EGM data, the training dataset would comprise sample EGM waveform segments collected from a population. These EGM samples would be annotated by medical experts to label each segment with the type of arrhythmia it represents, if any. The model would then be trained using this annotated EGM training dataset to map the input signals to the expert-provided arrhythmia labels using a supervised learning approach.

In contrast, a wearable cardiac patch monitor placed on the skin surface collects electrocardiogram (ECG) waveforms rather than direct cardiac electrical data. Although less detailed than EGM, ECG signals also contain patterns indicative of different arrhythmia types. Therefore, to train a machine learning model for a wearable ECG monitor, the training data would consist of sample ECG waveform segments annotated by medical experts. The model would be trained to classify arrhythmias based on the ECG morphology and timing features rather than EGM.

Accordingly, the training data used to develop an arrhythmia detection machine learning model needs to match the type of physiological signal data collected by the ambulatory medical device. This allows the model to reliably analyze the patterns and features relevant to that specific signal modality. The model is tuned to the characteristics of EGM versus ECG data based on using the appropriate annotated training dataset.

Once trained, the machine learning model can receive new ECG signal data as input and automatically detect if the patterns reflect normal sinus rhythm or different arrhythmia morphologies. This allows advanced arrhythmia analysis without needing to hard-code detection criteria. The model can be periodically retrained on new data to improve accuracy over time.

In the bottom portion of FIG. 3, the inference stage is presented. For example, after the machine learning model has been trained to reliably generate outputs that accurately predict the presence of arrythmia episodes in a training dataset and/or an evaluation dataset, the trained machine learning model can be deployed for use in a production setting. Accordingly, when physiological signal data 306 obtained by an ambulatory medical device is received at a remote server computer, the data 306 is provided as input to the trained machine learning model 304-A. The trained machine learning model generates one or more outputs, which are then interpreted to determine whether the input data represent an arrythmia episode.

In some examples, when the input data is determined to represent a specific type of arrythmia episode, a data record may be updated to reflect the identification of the arrythmia episode, along with various other relevant data items, such as, for example, the type of arrythmia event, the day and time at which the relevant physiological signal data was obtained by the ambulatory medical device, attributes of the device that obtained the physiological signa data, for example, such as the sensitivity setting at which the device was operating. As described in greater detail below, these patient-specific data records can be used as input to a rule-based recommendation engine for generating a recommendation to reprogram a medical device, for example, when one or more arrythmia episodes are detected by the machine learning analysis, but are not detected by the on-device arrythmia detection algorithms.

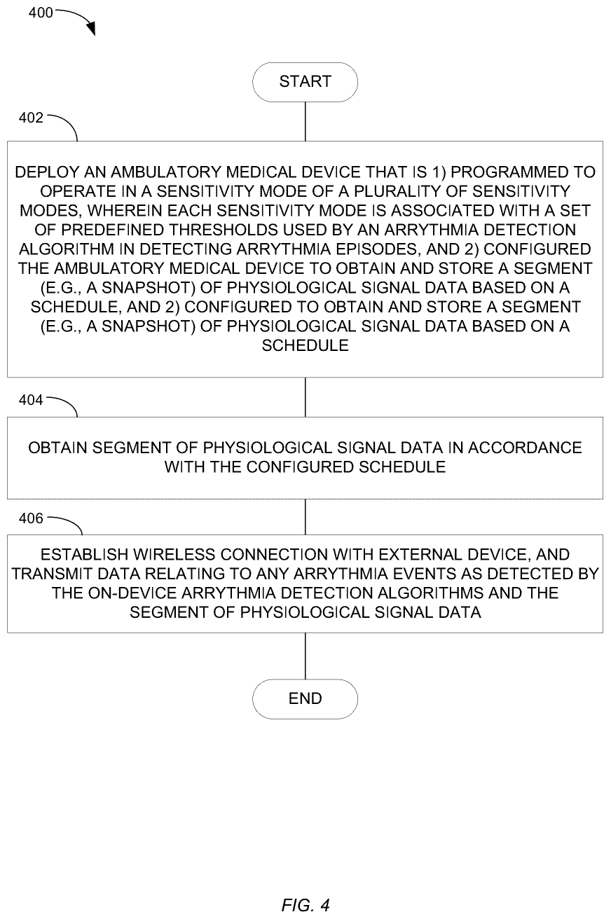

FIG. 4 is a flow diagram illustrating method operations for a method 400, performed, in part, by a medical ambulatory device, to obtain and transmit physiological signal data for analysis by a remote computer system, according to some embodiments. The method begins at operation 402, when a clinician programs and deploys an ambulatory medical device. For example, for an implantable ambulatory medical device like a cardiac monitor, deployment involves a surgical procedure to insert the device under the patient's skin. Deployment of a wearable ambulatory device such as a patch monitor requires preparation of the patient's skin where the device will be adhered. As part of deploying the medical device, the clinician will program the device to operate in one of a number of sensitivity modes, where each sensitivity mode is associated with a set of thresholds used by an arrythmia detection algorithm to detect arrythmia episodes. For example, many ambulatory medical devices like implantable cardiac monitors allow clinicians to configure or program the device to operate in one of several predefined sensitivity modes for detecting different arrhythmia types. During initial programming, the clinician selects a sensitivity mode based on factors like the patient's cardiac history and risk profile. Each sensitivity mode corresponds to a specific set of detection algorithm thresholds tailored to that mode. For example, a “high” sensitivity mode may have lower thresholds for beat irregularity, noise levels, and R-R interval variation compared to a “low” sensitivity mode, allowing more subtle arrhythmia episodes to be detected at the cost of reduced specificity.

Additionally, when initially deployed, the clinician may configure or program the device to periodically capture and store a segment—for example, a snapshot—of physiological signal data, such as thirty seconds of cardiac electrical data once per day, independent of any triggering event identified by the on-device detection algorithms. This scheduled background sampling allows segments of raw signal data to be collected over time. The sampled data is temporarily stored in the devices internal memory until it can be extracted. Accordingly, at method operation 404, the ambulatory medical device obtains and stores the segment of physiological signal data.

Next, at method operation 406, when the device establishes a wireless connection with an external device, such as an intermediary device like a mobile phone executing a dedicated mobile app, the stored physiological data segments are transmitted from the ambulatory medical device to the intermediary device. The mobile app then forwards the data over the Internet to a remote server in a secure cloud environment for further analysis. This method is repeated each day

On the remote server, the transferred signal data can be input into one or more machine learning models that have been pre-trained to identify different arrhythmia morphologies. The machine learning analysis may detect subtle arrhythmia episodes in the sampled data that were missed by the device's real-time detection algorithms. This deeper analysis by more advanced algorithms enables closed-loop optimization by identifying opportunities to adjust the device's detection parameters over time, such as recommending the clinician switch to a higher sensitivity mode if episodes are being under-detected.

The remote server provides a dashboard for clinicians to review the transmitted physiological data and remotely monitor patient status. Data associated with any arrhythmia events flagged by the on-device detection algorithms is also extracted and transmitted to the remote server to give greater clinical context.

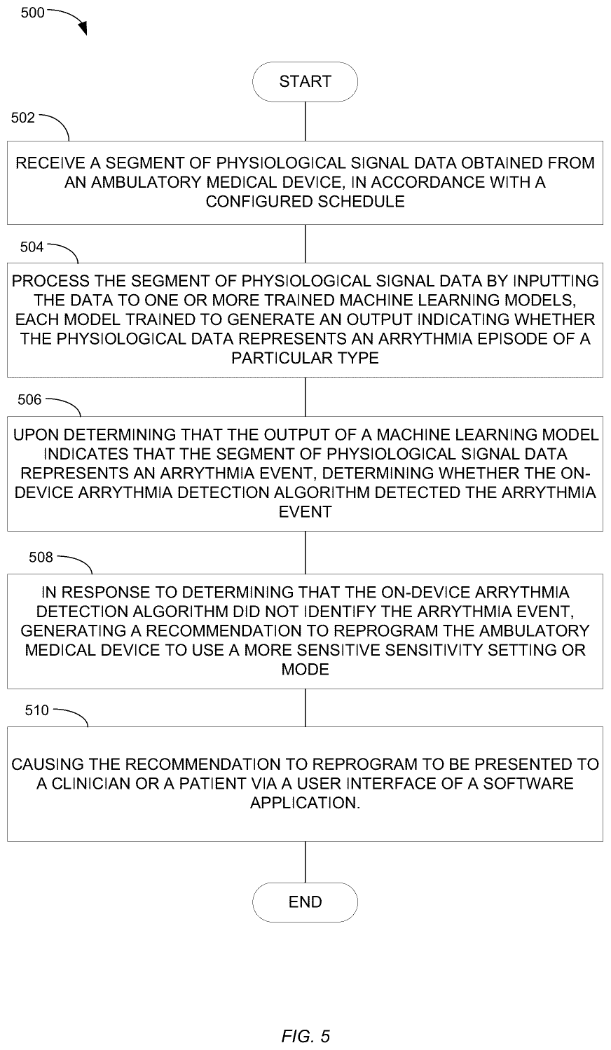

FIG. 5 is a flow diagram illustrating method operations for a method 500, performed by a computer system, to generate a reprogramming recommendation for an ambulatory medical device, according to some embodiments. The method begins at method operation 502 by a remote server computer receiving a segment of physiological signal data that was obtained from an ambulatory medical device on a periodic, scheduled basis. For example, the device can be configured to capture snapshots of raw signal data at regular intervals, unrelated to arrhythmia detection.

At method operation 504, this sampled physiological data is provided as input to one or more machine learning models that have been trained to detect different types of arrhythmias. For example, the models may include deep neural networks trained on annotated ECG data. The models analyze the physiological signal data and generate one or more outputs indicating whether the data contains any arrhythmia episode. If a model detects an arrhythmia that was missed by the device's real-time arrythmia detection algorithms, the system determines the device's detection algorithms are underperforming.

In response, at method operation 508, the system automatically generates a recommendation to reprogram the ambulatory medical device to operate at a more sensitive detection setting or mode, for instance, by lowering the thresholds required to declare an arrhythmia episode.

Finally, at method operation 510, the reprogramming recommendation is presented to a clinician or patient via a software application interface to optimize future arrhythmia detection. Accordingly, based on the reprogramming recommendation, a clinician may reprogram the device to operate at a different sensitivity setting or mode.

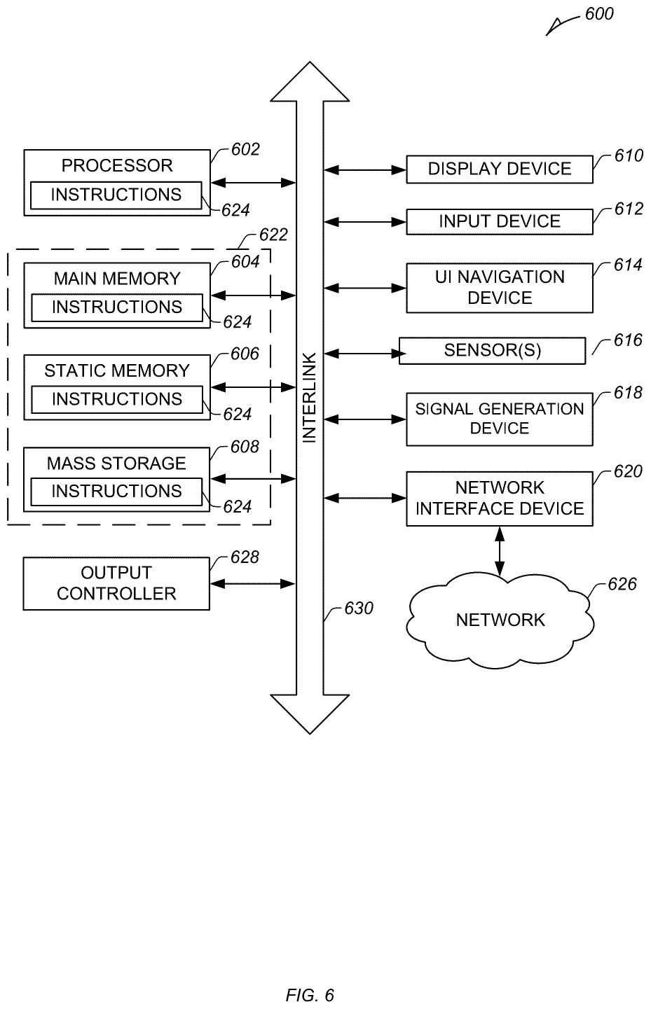

FIG. 6 illustrates a block diagram of an example machine 600 upon which any one or more of the techniques (e.g., methodologies) discussed herein may perform. Portions of this description may apply to the computing framework of one or more of the medical devices described herein, such as the implantable medical device, the external programmer, and so forth. Further, as described herein with respect to medical device components, systems, or machines, such may require regulatory-compliance not capable by generic computers, components, or machinery.

Examples, as described herein, may include, or may operate by, logic or a number of components, or mechanisms in the machine 600. Circuitry (e.g., processing circuitry, an assessment circuit, etc.) is a collection of circuits implemented in tangible entities of the machine 600 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 600 follow.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 600 (e.g., computer system) may include a hardware processor 602 (e.g., a central processing unit (CPU), a graphics processing unit (GPU), a hardware processor core, or any combination thereof), a main memory 604, a static memory 606 (e.g., memory or storage for firmware, microcode, a basic-input-output (BIOS), unified extensible firmware interface (UEFI), etc.), and mass storage 608 (e.g., hard drive, tape drive, flash storage, or other block devices) some or all of which may communicate with each other via an interlink 630 (e.g., bus). The machine 600 may further include a display unit 610, an input device 612 (e.g., a keyboard), and a user interface (UI) navigation device 614 (e.g., a mouse). In an example, the display unit 610, input device 612, and UI navigation device 614 may be a touch screen display. The machine 600 may additionally include a signal generation device 618 (e.g., a speaker), a network interface device 620, and one or more sensors 616, such as a global positioning system (GPS) sensor, compass, accelerometer, or one or more other sensors. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 602, the main memory 604, the static memory 606, or the mass storage 608 may be, or include, a machine-readable medium 622 on which is stored one or more sets of data structures or instructions 624 (e.g., software) embodying or utilized by any one or more of the techniques or functions described herein. The instructions 624 may also reside, completely or at least partially, within any of registers of the hardware processor 602, the main memory 604, the static memory 606, or the mass storage 608 during execution thereof by the machine 600. In an example, one or any combination of the hardware processor 602, the main memory 604, the static memory 606, or the mass storage 608 may constitute the machine-readable medium 622. While the machine-readable medium 622 is illustrated as a single medium, the term “machine-readable medium” may include a single medium or multiple media (e.g., a centralized or distributed database, and/or associated caches and servers) configured to store the one or more instructions 624.

The term “machine-readable medium” may include any medium that is capable of storing, encoding, or carrying instructions for execution by the machine 600 and that cause the machine 600 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may be further transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 600, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A computing device for generating a reprogramming recommendation for an ambulatory medical device, the computing device comprising:
one or more processors; and
a memory device storing instructions, which when executed by the processor, cause the computing device to perform operations comprising:
receiving over a network physiological signal data obtained by the ambulatory medical device;
processing the received physiological signal data by inputting the physiological signal data into one or more pre-trained machine learning models, each of the one or more pre-trained machine learning models trained to generate an output indicating whether the physiological signal data represents an arrhythmia episode of a particular type;
upon obtaining an output from the one or more pre-trained machine learning models indicating a detected arrythmia episode of a first type, determining that an on-device arrythmia detection algorithm of the ambulatory medical device did not detect a corresponding arrythmia episode of the first type; and
as a result of determining that the on-device arrythmia detection algorithm of the ambulatory medical device did not detect a corresponding arrythmia episode of the first type, generating the reprogramming recommendation for the ambulatory medical device.

2. The computing device of claim 1, wherein the ambulatory medical device is programmed to operate in a first sensitivity mode of a plurality of sensitivity modes with each sensitivity mode corresponding with a first set of predefined threshold values for use by an on-device arrythmia detection algorithm in detecting arrythmia episodes, and the reprogramming recommendation is a recommendation to program the ambulatory medical device to operate using a second sensitivity mode, the second sensitivity mode having at least one predefined threshold value for use by the on-device arrythmia detection algorithm that is lower than a corresponding predefined threshold value for the first sensitivity mode, thereby providing increased sensitivity for detecting arrhythmia episodes compared to the first sensitivity mode.

3. The computing device of claim 2, wherein the on-device arrythmia detection algorithm of the ambulatory medical device operates in two stages comprising a detection stage and a confirmation stage;
wherein i) during the detection stage, the on-device arrythmia detection algorithm analyzes incoming physiological signal data and identifies candidate arrythmia events based on the first set of predefined threshold values associated with the first sensitivity mode, ii) upon identifying a candidate arrythmia event during the detection stage, the ambulatory medical device begins recording and storing a segment of the physiological signal data corresponding to the time of the initial candidate arrythmia event detection, iii) during the confirmation stage, the on-device arrythmia detection algorithm analyzes the stored segment of physiological signal data corresponding to the candidate arrythmia event to confirm whether the event meets criteria for a confirmed arrythmia episode based on the first set of predefined detection algorithm threshold values associated with the first sensitivity mode, and iv) changing the programming of the ambulatory medical device from the first sensitivity mode to the second sensitivity mode results in changing to a second set of predefined detection algorithm threshold values used by one or both of the detection stage and confirmation stage.

4. The computing device of claim 1, wherein the physiological signal data obtained by the ambulatory medical device is stored locally on the ambulatory medical device until a wireless connection is established between the ambulatory medical device and an intermediary device, and upon establishing the wireless connection with the intermediary device, the physiological signal data is transmitted from the ambulatory medical device to the intermediary device and then transmitted from the intermediary device to the computing device over a network.

5. The computing device of claim 1, wherein processing the received physiological signal data comprises:
inputting the physiological signal data into a trained machine learning model to generate by the trained machine learning model a plurality of confidence scores, each confidence score indicating a likelihood that the physiological signal data represents an arrhythmia episode of a particular type of arrythmia; and
for a first type of arrythmia, comparing the confidence score for the first type of arrhythmia to a confidence threshold to determine whether an episode of the first arrhythmia type is detected in the physiological signal data.

6. The computing device of claim 1, wherein processing the received physiological signal data comprises:
inputting the physiological signal data into each of a plurality of trained machine learning models, each trained machine learning model trained to detect a different arrhythmia type;
obtaining an output from each of the plurality of trained machine learning models indicating whether the physiological signal data represents an episode of the arrhythmia type the model is trained to detect; and
determining that the physiological signal data represents an episode of the first arrhythmia type based on the output of the one of the plurality of trained machine learning models trained to detect the first arrhythmia type.

7. The computing device of claim 1, wherein the first type of arrhythmia episode is an atrial fibrillation episode and the reprogramming recommendation is a recommendation to reprogram the ambulatory medical device to use a sensitivity setting having predefined threshold values for an on-device arrythmia detection algorithm that are more sensitive for detecting atrial fibrillation episodes.

8. The computing device of claim 7, wherein increased sensitivity for detecting atrial fibrillation episodes by the on-device arrythmia detection algorithm is achieved by modifying one or more of:
decreasing an R-R interval irregularity threshold used by the ambulatory medical device to declare an atrial fibrillation episode;
decreasing a density index threshold calculated from R-R intervals that must be satisfied to declare an atrial fibrillation episode;
decreasing a minimum atrial fibrillation episode duration threshold.

9. The computing device of claim 1, wherein the output from the one or more machine learning models indicates that the physiological signal data contains an arrythmia episode comprising one of premature ventricular contractions (PVCs) or premature atrial contractions (PACs); and
based on the indication of PVCs/PACs in the physiological signal data, the reprogramming recommendation comprises modifying one or more predefined threshold values used by an on-device arrythmia detection algorithm of the ambulatory medical device to detect an episode of PVC/PAC.

10. The computing device of claim 1, wherein the output from the one or more machine learning models indicates that the physiological signal data contains T-wave oversensing (TWOS); and
based on the indication of TWOS in the physiological signal data, the reprogramming recommendation comprises a recommendation to program the ambulatory medical device to operate with a sensitivity mode having a different refractory period to avoid oversensing of T-waves.

11. The computing device of claim 1, wherein generating the reprogramming recommendation comprises:
determining that the output from the one or more machine learning models indicates detection of the arrhythmia episode of the first type a predetermined plurality of times;
wherein the reprogramming recommendation is generated only after the predetermined plurality of times that the arrhythmia episode of the first type is detected by the one or more machine learning models.

12. The computing device of claim 1, wherein generating the reprogramming recommendation comprises:
incrementing a counter each time the output from the one or more machine learning models indicates detection of the arrhythmia episode of the first type; and
determining that the counter exceeds a threshold number of days, wherein the counter indicates detection of the arrhythmia episode of the first type on consecutive days;
wherein the reprogramming recommendation is generated only after the counter indicates that the arrhythmia episode of the first type is detected on the consecutive days.

13. A method comprising:
receiving over a network physiological signal data obtained by an ambulatory medical device;
processing the received physiological signal data by inputting the physiological signal data into one or more pre-trained machine learning models, each of the one or more pre-trained machine learning models trained to generate an output indicating whether the physiological signal data represents an arrhythmia episode of a particular type;
upon obtaining an output from the one or more pre-trained machine learning models indicating a detected arrythmia episode of a first type, determining that an on-device arrythmia detection algorithm of the ambulatory medical device did not detect a corresponding arrythmia episode of the first type; and
as a result of determining that the on-device arrythmia detection algorithm of the ambulatory medical device did not detect a corresponding arrythmia episode of the first type, generating a reprogramming recommendation for the ambulatory medical device.

14. The method of claim 13, wherein the ambulatory medical device is programmed to operate in a first sensitivity mode of a plurality of sensitivity modes with each sensitivity mode corresponding with a first set of predefined threshold values for use by an on-device arrythmia detection algorithm in detecting arrythmia episodes, and the reprogramming recommendation is a recommendation to program the ambulatory medical device to operate using a second sensitivity mode, the second sensitivity mode having at least one predefined threshold value for use by the on-device arrythmia detection algorithm that is lower than a corresponding predefined threshold value for the first sensitivity mode, thereby providing increased sensitivity for detecting arrhythmia episodes compared to the first sensitivity mode.

15. The method of claim 14, wherein the on-device arrythmia detection algorithm of the ambulatory medical device operates in two stages comprising a detection stage and a confirmation stage;
wherein i) during the detection stage, the on-device arrythmia detection algorithm analyzes incoming physiological signal data and identifies candidate arrythmia events based on the first set of predefined threshold values associated with the first sensitivity mode, ii) upon identifying a candidate arrythmia event during the detection stage, the ambulatory medical device begins recording and storing a segment of the physiological signal data corresponding to the time of the initial candidate arrythmia event detection, iii) during the confirmation stage, the on-device arrythmia detection algorithm analyzes the stored segment of physiological signal data corresponding to the candidate arrythmia event to confirm whether the event meets criteria for a confirmed arrythmia episode based on the first set of predefined detection algorithm threshold values associated with the first sensitivity mode, and iv) changing the programming of the ambulatory medical device from the first sensitivity mode to the second sensitivity mode results in changing to a second set of predefined detection algorithm threshold values used by one or both of the detection stage and confirmation stage.

16. The method of claim 13, wherein the physiological signal data obtained by the ambulatory medical device is stored locally on the ambulatory medical device until a wireless connection is established between the ambulatory medical device and an intermediary device, and upon establishing the wireless connection with the intermediary device, the physiological signal data is transmitted from the ambulatory medical device to the intermediary device and then transmitted from the intermediary device to a computing device over a network.

17. The method of claim 13, wherein processing the received physiological signal data comprises:
inputting the physiological signal data into a trained machine learning model to generate by the trained machine learning model a plurality of confidence scores, each confidence score indicating a likelihood that the physiological signal data represents an arrhythmia episode of a particular type of arrythmia; and
for a first type of arrythmia, comparing the confidence score for the first type of arrhythmia to a confidence threshold to determine whether an episode of the first arrhythmia type is detected in the physiological signal data.

18. The method of claim 13, wherein processing the received physiological signal data comprises:
inputting the physiological signal data into each of a plurality of trained machine learning models, each trained machine learning model trained to detect a different arrhythmia type;
obtaining an output from each of the plurality of trained machine learning models indicating whether the physiological signal data represents an episode of the arrhythmia type the model is trained to detect; and
determining that the physiological signal data represents an episode of the first arrhythmia type based on the output of the one of the plurality of trained machine learning models trained to detect the first arrhythmia type.

19. The method of claim 13, wherein the first type of arrhythmia episode is an atrial fibrillation episode and the reprogramming recommendation is a recommendation to reprogram the ambulatory medical device to use a sensitivity setting having predefined threshold values for an on-device arrythmia detection algorithm that are more sensitive for detecting atrial fibrillation episodes.

20. The method of claim 19, wherein increased sensitivity for detecting atrial fibrillation episodes by the on-device arrythmia detection algorithm is achieved by modifying one or more of:
decreasing an R-R interval irregularity threshold used by the ambulatory medical device to declare an atrial fibrillation episode;
decreasing a density index threshold calculated from R-R intervals that must be satisfied to declare an atrial fibrillation episode; and
decreasing a minimum atrial fibrillation episode duration threshold.
